(19) United States
(12) Patent Application Publication
(10) Pub. No.: US 2024/0221952 A1
(43) Pub. Date: Jul. 4, 2024

(54) DATA-BASED CLINICAL DECISION-MAKING UTILISING KNOWLEDGE GRAPH

(71) Applicant: Smart Reporting GmbH, Munich (DE)

(72) Inventors: Su Hwan KIM, Munich (DE); Francisco PINTO, Munich (DE); Alvaro Sanchez MOSCOSA, Munich (DE); Vlad LAZAR, Munich (DE)

(21) Appl. No.: 18/579,919

(22) PCT Filed: Jul. 21, 2021
(86) PCT No.: PCT/EP2021/070449

§ 371 Date: Jan. 17, 2024

(51) Int. Cl. G16H 50/20; G06F 16/901; G16H 15/00

(52) U.S. Cl. CPC G16H 50/20; G06F 16/9024; G16H 15/00

(57) ABSTRACT

A method, a computer system, and a computer program product are provided for supporting data-based clinical decision-making. Medical concepts and relations between medical concepts contained in a structured medical report and/or a template for such reports are extracted. The template and/or the structured medical report comprise a data structure representing the medical concepts and relations between these medical concepts. These extracted medical concepts and relations between the medical concepts are then integrated into a graph database and weighted according to their relevance. Based on the weights one or more recommendations for actions a user may take when composing report templates and/or structured medical reports are inferred and presented to the user.

This invention pertains to the field of creating medical reports and creating templates for such reports.

### BACKGROUND OF THE INVENTION

When generating a medical report, the physician summarizes her or his observations, e.g. during anamnesis or when reviewing medical images of a patient. A typical report is created item by item. E.g., in a radiological report of a CT of the thorax the radiologist is reporting organ by organ, e.g., looking, beyond others, at the heart, pericardium, lung parenchyma and airways. In a subsequent step, the reporting physician summarizes the main findings into an impression. The main findings of a report contain so-called key findings which indicate remarkable aspects.

The report can be free-text. Then, its structure, elements, style, wording and layout may differ from physician to physician. They are not machine-readable, not standardized, and not analyzable. Moreover, they are prone to artefacts and they might be unclear or even incomplete.

To overcome the drawbacks of free-text reports, so-called structured medical reports were introduced. These are based on structured, machine-readable reporting templates that can be progressively filled in by reporting physicians. Ideally, a structured report is machine-readable, has a fixed structure and contains standardized elements, wording and layout. In addition, pre-generated report templates can be used. These may provide case-specific structure.

An example for an implementation of a structured report is illustrated in WO 2016/135100 A1. There an approach to provide structured reports is described, which proposes the use of report modules as report building blocks based on decision trees, which allows to add or remove report modules to a report.

In addition to using pre-generated report templates a user has the possibility to create his/her own report templates. The user can then use the templates he/she has created and adapted to his/her needs when reporting and/or share the templates with other users, e.g. via direct exchange or an expert-reviewed database of templates.

In order to ensure semantic interoperability—be it between machines or humans, structured reports can be annotated with medical terminologies and/or ontologies. Medical terminologies/ontologies help to standardize the capture, storage, exchange and unambiguous interpretation of clinical data across multiple applications.

A medical terminology is a collection of terms that are used in the field of medicine, or in a particular sub-domain, subject, or specialty. It serves to facilitate communication amongst humans and communication between human and machine.

Ontologies are sets of concepts in the respective medical subject area that assign unambiguous meaning and define relations between concepts. Thus, an ontology may provide terminology, but also represents a knowledge domain by reflecting the properties of the domains terms and/or concepts and the relations between them. Further, ontologies may use ontology specific codes as identifiers to unambiguously encode the objects and relations. Thus, it obtains a systematic correlation between reality and its representation in a way that it is intelligible to a domain expert while being formalized in a way that allows it to support automatic information processing. It can be understood as a representation of “reality” inside a machine and therefore enable communication amongst machines. For simplification, all structured medical terminologies are often referred to as “medical ontologies”. In the following, the term “medical ontologies” will be used to describe structured medical terminologies in general. Medical ontologies are known for a variety of medical subdomains, including genetics, adverse events, anatomy, drugs, diseases, and medical codes, e.g. RadLex or SNOMED CT.

However, expanding ontologies, e.g., by adding translations, new concepts, new synonyms, new relations or mappings of one ontology to another is complex. Extensions are predominantly implemented through a highly sophisticated change request submission process which requires authorized stakeholders to dedicate time and effort.

Further, ontologies do not accurately reflect the usefulness and relevance of its elements in clinical practice.

Finally, applying the full concept model of large medical ontologies in the form of relational databases or object-oriented databases—the commonly used database architectures in electronic health record applications—has proven highly challenging due to their size and complexity.

Clinical decision-making, both behind the actual reporting and in preparatory steps such as the preparation of report templates, is often very complex; many aspects and necessary steps must be considered in order to meet high clinical quality standards. Therefore, it would be of great help to support and guide reporting physicians based on existing medical knowledge, in particular medical concepts and relations between these concepts, as well as on clinical data.

This could accelerate clinical workflows by ensuring the completeness of the necessary information while at the same time improving the quality of their results by avoiding erroneous entries and/or overlooking critical aspects.

### SUMMARY OF THE INVENTION

The purpose of this invention is to improve data-based clinical decision-making.

The invention provides a method, a system and a computer program product for improving the support of data-based clinical decision-making, in particular when preparing and/or carrying out clinical reporting.

According to one aspect of the invention a computer-implemented method for supporting data-based clinical decision-making is provided, comprising:
a. extracting medical concepts and relations between medical concepts contained in a structured medical report and/or a template for such reports, the template and/or the structured medical report comprising a data structure representing the medical concepts and relations between these medical concepts;
b. integrating the extracted medical concepts and relations between the medical concepts into a graph database;
c. weighting the medical concepts and relations between these medical concepts of the graph database according to their relevance;
d. based on the weights of the medical concepts and relations between these medical concepts stored in the graph database, inferring and presenting to a user one or more recommendations for actions the user may take when composing report templates and/or structured medical reports;

According to another aspect of the invention a computer system for supporting data-based clinical decision-making is provided, comprising:
a first data storage storing at least one structured medical report and/or a template for such reports, the template and/or the structured medical report comprising a data structure representing medical concepts and relations between the medical concepts;
a data extractor that extracts medical concepts and relations between medical concepts contained in the stored structured medical report and/or the template for such reports;
a graph database manager that
   integrates the extracted medical concepts and relations between the medical concepts into a graph database stored to the first or a second data storage; and
   weights the medical concepts and relations between the medical concepts integrated into the graph database;
a recommender that infers and presents to a user one or more recommendations for actions the user may take, based on the weights of the medical concepts and relations between these medical concepts stored in the graph database; and
an output unit that presents the inferred recommendations to the user.

According to another aspect of the invention a computer program product for supporting data-based clinical decision-making is provided, comprising computer readable instructions to execute the steps of the methods described here.

In the following, aspects and embodiments of the invention will be discussed. The details and resulting advantages of the invention will appear from the following description. Where appropriate, reference is made to the corresponding drawings, which show preferred embodiments of the invention for illustration purposes. However, these embodiments do not necessarily represent the full scope of the invention.

### BRIEF DESCRIPTION OF THE DRAWINGS

FIG. 1 is a flow chart illustrating a method according to the invention;

FIG. 2 illustrates the relation between structured medical reports and their templates;

FIG. 3 illustrates the expansion of a graph database based on structured medical reports and templates for such reports;

FIGS. 4 to 10 are flowcharts illustrating several implementations according to embodiments of the invention;

FIG. 11 shows a computer system according to an embodiment of the invention.

Graph database technology has been shown to be extremely powerful in managing and querying huge, complex datasets and has been applied in multiple other industries (e.g. social media, search engines) for big data analytics. In their simplest form, the elements of graph databases consist of concepts and relations between those concepts. A graph database can be directed or undirected.

An example of a graph database is a so-called knowledge graph, in which the concepts and their relations between each other represent the knowledge of a specific knowledge domain, e.g. medicine.

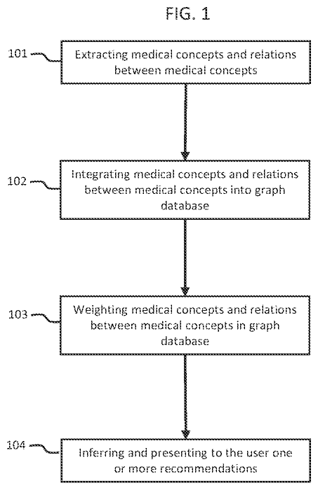

One aspect of the invention is illustrated in FIG. 1 and based on the idea of integrating medical concepts and relations between these concepts in such a graph database, i.e. to store and organize them in this form. For this purpose, medical concepts and relations between concepts, are extracted from structured medical reports and/or templates for such structured medical reports 101 and integrated into the graph database 102. For example, this way a knowledge graph representing the knowledge of at least one subdomain of medicine may be built.

An advantage of extracting 101 these data from structured medical reports or the templates of such reports is that the data structure behind them represents the elements of the graph database, i.e. in the simplest case the concepts and relations between the concepts. In other words, the elements that form the graph database are comprised as such in a structured report and its template. Therefore, these elements can easily be transferred from one data structure to the other and be integrated into the graph database. E.g., the concepts and relations between them may be extracted in form of triples which can then be directly integrated into the graph database. A triple in general can be defined as a set of three entities that represent a code for a statement about semantic data. In particular, a triple may consist of two concepts and a relation between these two concepts. By doing so, the graph database is continuously expanded and optimized based on medical reports or report templates created by users.

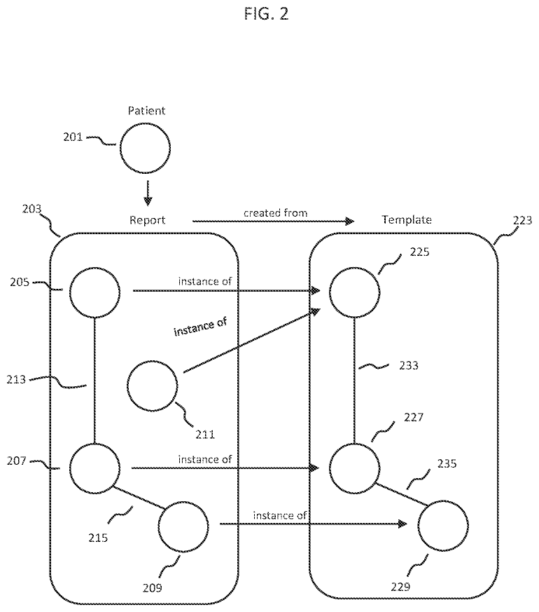

There are many possible implementations of templates, reports and how they are linked to each other. For example, in some embodiments the abstract concepts of the template may be represented as nodes (e.g. of a tree structure). These may be identified by a unique ontology code (which is discussed later in this text). In contrast, report nodes form instances of those concepts that represent real-world clinical data and can occur one or more multiple times (cardinality). The abstract concepts of the template may therefore be linked to one or more instances of the concept within the report. For example, the concept “lung tumor” could occur in multiple reports, and even multiple times within a single report to represent the observation of several lung tumors in a CT scan. Using unique node ids/codes, e.g. ontology codes, allows the unambiguous identification of every concept instance. In addition, reports can further be linked to a patient node with patient id/gender/date of birth/etc. as possible properties. FIG. 2 illustrates this relation between the templates and the reports. A patient 201 has a report 203 comprising several concepts 205-211, and relations 213 and 215 between these concepts. The template 223 comprises concepts 225-229 and relations 233 and 235 between them. The real-life concepts 205-211 are instances of the concepts 225-229.

An alternative or additional implementation would be, for example, to store the reports in a separate database and to perform a federated query over two or more databases for the data evaluations.

When integrating the concepts and relationships into the graph database 102, two cases are conceivable. In the first case, the concepts and/or relationships to be developed are not yet contained in the graph database. In this case it is possible to add the corresponding entries to the database. In the second case the concepts and/or relations to be integrated are already known, i.e. they already exist in the graph database. In this case, the existing entries are updated and/or optimized if necessary.

In addition, the elements of the graph database can be weighted 103. Ideally this is done using data reflecting the practical usefulness of the individual concepts and relations, e.g. in clinical routine or a specific clinical or user context. Thus, these weightings may represent measures for the clinical relevance of the individual graph database entries. They may be predefined, user defined, or be derived from data—e.g., the data extracted from the reports or templates. The weighting of the entries can be done during or after the integration 102 of the concepts and relations into the graph database.

Based on this weighting, recommendations can be inferred and be provided to the user 104. These can be recommendations useful when creating a report template and/or recommendations useful when creating a report. Such recommendations can, for example, be recommendations for further actions or steps the user may be required to or choose to take. In general, recommendations include, beyond others, specific instructions to the user, suggestions for further steps, suggestions for elements to be included in a report or a template, terminology-suggestions, suggestions to consult background information and/or guidelines, and/or suggestions to include or request further data.

These inferred recommendations and/or instructions are then presented to the user. This can be done at appropriate positions or times during the creation of a structured medical report and/or a template for such reports. The user may be presented with such recommendations via visual and/or auditory channels. For example, a screen or other display device can be used to present recommendations visually. The user is then given the inferred recommendations and/or indications in the form of pop-ups or other windows or info boxes that can be displayed during his/her work. Additionally or alternatively, certain elements and/or locations of the reports or templates can be highlighted to draw the user's attention to them; colored underlays, altered text color, and/or altered typeface are examples here. As an alternative or in addition to the visual channel, auditory recommendations can also be given to the user. This may simply be an audio signal that indicates to the user that visually presented recommendations appeared on a display device. However, it is also possible to present the recommendations to the user completely acoustically. This can be useful, for example, if the user cannot or can only rarely look at a screen while working.

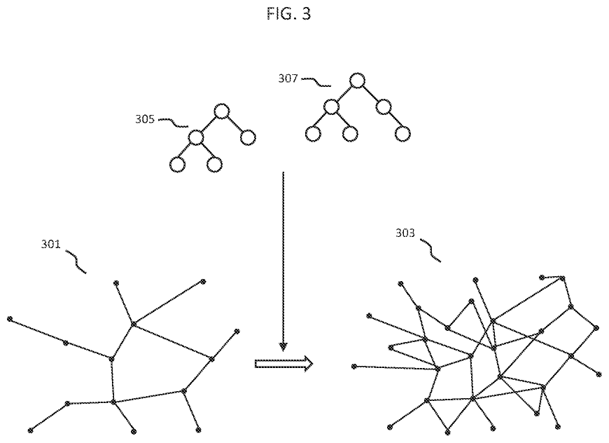

This significantly reduces the effort required to expand existing medical graph databases by linking the composition of structured report templates to medical graph database expansions. These report templates are created to capture clinical patient data in a structured manner and feeding back the concepts, terms and relationships applied within a template does not entail any further effort for the user. FIG. 3 illustrates the expansion of the existing graph database 301 to the expanded graph database 303 based on structured medical reports and/or templates for such a report 305 and 307.

In this way, a self-reinforcing feedback loop is created in which the graph database is expanded, updated, and/or optimized based on clinical data and the quality of the collected clinical data is ultimately improved via recommendations based on knowledge stored in the graph database. As the graph database and the library of annotated templates and reports grows, the recommendation system is permanently optimized.

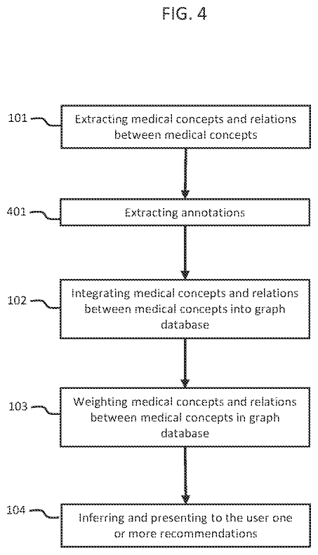

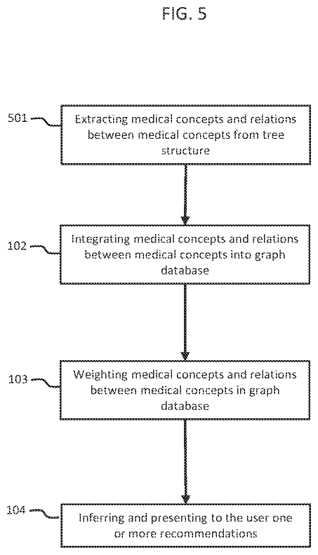

One embodiment that is illustrated in FIG. 4 comprises extracting 401 from a template for structured medical reports and/or a structured medical report annotations of the medical concepts and relations between the medical concepts. These annotations are, e.g., be based on a medical ontology.

In fact, graph databases might be particularly useful in light of the polyhierarchical design of many medical ontologies. Thus, in one embodiment, the graph database can be a representation of an existing version of an ontology and the graph database is continuously complemented by expert-approved and ontology-annotated concepts from newly created templates and/or structured medical reports. Using ontology-annotated report templates and structured reports allows to recognize semantically equivalent terms and expressions in the reports. E.g., the expressions “lung tumor” and “tumor located in the lung” means semantically the same and will therefore be annotated with the same identifiers of an ontology. Thus, annotating the entries of the graph database using ontology-identifiers allows to recognize semantically equivalent concepts and relations.

Furthermore, the present method also allows to assign multiple unique identifiers from multiple different ontologies to one entry of the database. E.g., the entries of the graph database may themselves be annotated with identifiers from several different ontologies. Thus, the graph database can be ontology agnostic in the sense that it is able to correctly interpret annotations based on different ontologies. Based on the database, different ontology codes that are, however, semantically equivalent can be easily recognized. For instance, the concept “Lung cancer” can be represented through the RadLex code RID45686 or the SNOMED CT code SCTID: 93880001. Based on the graph database both can be recognized as referring to the same clinical concept. This has several advantages. First, the graph database can be extended and/or optimized extracting elements of report templates independent of what ontology was used to annotate the entries of the data structure behind the template or report. Second, the graph database can be used for ontology mapping, i.e. for mapping the identifiers of one ontology to another. Third, templates and/or structured reports that are composed based on the graph database can be annotated with identifiers of one or more of several different ontologies. The user might, e.g. select which ontology/ontologies to use.

Once an ontology code (unique identifier) has been assigned to an entry of a template or of a structured report, related concepts as represented in the graph database can be suggested to facilitate the creation of meaningful and logical templates and/or structured reports. These suggestions can be based on various recommendation systems taking into account multiple factors like, beyond others, proximity within the graph database, frequency of usage within annotated templates and reports.

For example, the annotation of template elements with unique identifiers can be (semi-) automated using machine learning-based medical concept annotation tools. One example of such an annotation tool is MedCAT (https://github.com/CogStack/MedCAT; open source). In essence, the tool is based on an algorithm mapping the terms of any kind of textual data to the concepts of existing ontologies. The annotation tool could as well be applied to the textual representation of the template in order to generate preliminary annotations which then can be reviewed by domain experts.

Thus, the graph database includes synonyms, translations, concepts and relationships, and the proposed methods and systems allow to add new synonyms, translations, concepts and relationships. When integrating concepts and/or relations to the graph database, a review and approval process of newly proposed content could further be supported and semi-automated through quality control mechanisms based on the graph database. For instance, the addition of redundant concepts could be prevented by searching through all preexisting concept synonyms within the graph database or relevant relationships to other concepts could be deduced from previous relationships within the graph. Moreover, newly introduced concepts could be filtered and reviewed based on their use during clinical routine reporting.

It should be noted, that in order to add translations or synonyms for terms of existing ontology concepts to one or more entries of the graph database, the respective concept might have to be identified using existing terms describing it.

Structured medical reporting templates serve as a tool to guide clinical documentation and capture highly valuable clinical data by providing a meaningful content structure. Such a reporting template and as a consequence the structured medical report based on such a template can have various data structures.

In some embodiments possible data structures representing medical concepts and relations between these medical concepts comprised in the template and/or the structured medical report include tables, graphs, linked lists or tree structures or a combination of one, some or all of these. In one embodiment illustrated in FIG. 5 the data structure representing medical concepts and relations between these medical concepts comprised in the template and/or the structured medical report is chosen 501 to have a tree structure.

In fact, the tree structure is one of the most common data structures for structured reports and their templates. A tree is a hierarchically organized data structure which consists of multiple aggregated nodes. Each node represents a semantic concept, out of which some may be part of existing medical ontologies and others not. Within a tree data structure, the relationship between two hierarchically organized nodes is referred to as a parent/child relationship (“node A is the parent node of node B”, “node B is the child node of node A”). One or more nodes form a data element which allows for data entry by the user. A data element can be characterized by its type of data input which may be one or more selections out of a predefined list or a text or number input by the user.

Given that a template tree consists of nodes and its parent-child relationships, the corresponding concepts and relations can be easily transferred into the graph database. If a parent node and its child node within the template or the report represent two medical concepts that do not have a relation (commonly referred to as “edge” in the field of data science) within the graph database yet, this new relationship is recognized as such. The type of relationship—which is a common parameter in medical ontologies—is also determined at this point of the template creation process. As already discussed, such new relationships can be added to the graph database as new entries.

In one embodiment, during the creation process of the structured template, unique identifiers of ontology concepts are assigned to individual nodes of the decision tree (=annotation, see above). Then, node labels introduced by the template author can be used for a fuzzy query within the terms of the annotated graph database. As described above, synonyms and translations are also covered by the terminology system of the graph database and can be matched to the entered node labels.

Depending on the design of the graph database, several additional entities that can be extracted from structured medical reports and/or templates for such reports and be integrated into the graph database can be assigned unique identifiers, in particular an edge (=relationship between two nodes), a node attribute, an edge attribute, a type of node and/or a type of edge.

Some embodiments comprise extracting the medical concepts and relations between these concepts in a specific format. E.g., one embodiment comprises extracting the medical concepts and relations between these concepts as triples. A triple consists of two concepts and the relation between the two concepts. These triples can form the basic building blocks of the graph database. Thus, extracting concepts and relations as triples can make it easier to integrate them into the database.

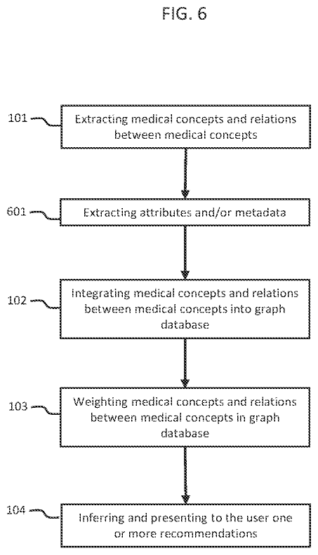

Some embodiments comprise extracting further information and/or data from the structured reports and/or report templates. This is illustrated in FIG. 6. One embodiment comprises extracting 601 also attributes of the medical concepts and/or relations between the concepts and/or metadata. These metadata may comprise a template ID, a report ID, patient ID, physician ID, institution ID, a time stamp, and/or an activation context.

The metadata can be used to provide additional information of the patient's case and the context which may be used to weight the entries of the graph database.

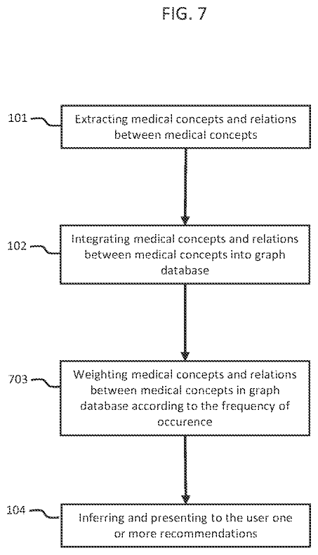

When it comes to weighting the elements of the database there are several options to implement the weighting which is illustrated in FIGS. 7-10.

In general, by linking the graph database to templates and reports generated using the templates, information about the usefulness and frequency of usage of concepts and relationships in clinical practice are immediately reflected in the database. This information can be used for determining the weights of the database entries.

For example, in one embodiment illustrated in FIG. 7, the concepts and relations in the graph database can be weighted 703 based on the frequency of occurrence of the individual annotated concepts within the templates for the structured medical reports and/or the corresponding structured medical reports created using those templates. For example, it can be counted how many times a concept or relation has been used in a report and/or report template and this number can be compared to the total number of all structured reports and/or report templates used so far for data extraction.

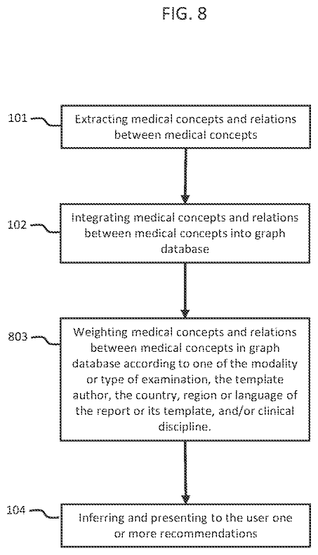

In other embodiments illustrated in FIG. 8, data that can be associated with the graph structure and be used for weighting its elements 803 are, e.g. the modality or type of examination, the template author, the country, region or language of the report or its template, and/or clinical discipline. The weighting can therefore be used as a measure of clinical relevance.

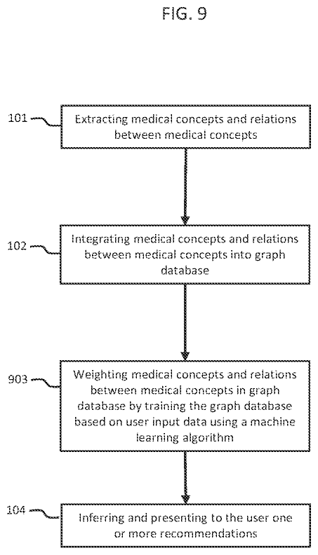

In yet another embodiment illustrated in FIG. 9, the weighting of the elements of the graph database comprises training 903 the knowledge graph based on user input data using a machine learning algorithm.

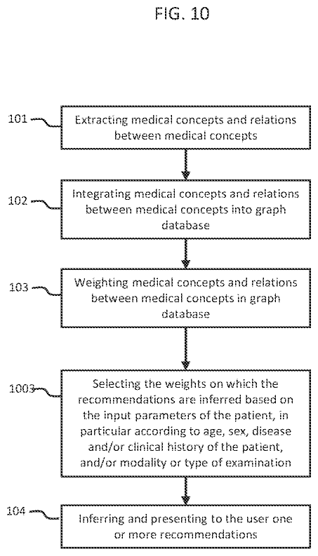

Further in some embodiments, the weighting can also be dynamically adapted to the context. For example, the possibilities for determining the weighting listed above as examples can also be further refined according to their clinical context. For example, the frequency of occurrence discussed earlier can be determined as the frequency of occurrence of a concept or relation in a particular clinical context. In one of these embodiments, such a context can be based on the input parameters of a patient. In particular the context can be based on age, sex, disease and/or clinical history of the patient, and/or modality or type of examination. This allows the providing of context-based recommendations to the user. For example, in one embodiment illustrated in FIG. 10 the weights on which the recommendations are inferred can be selected 1003 based on the input parameters of the patient, in particular according to age, sex, disease and/or clinical history of the patient, and/or modality or type of examination.

The recommendations that are inferred and provided to the user may be of several kinds and may depend on whether the user is creating a template or a report. In addition, recommendations may depend on the clinical context, the patient's case, and/or present data.

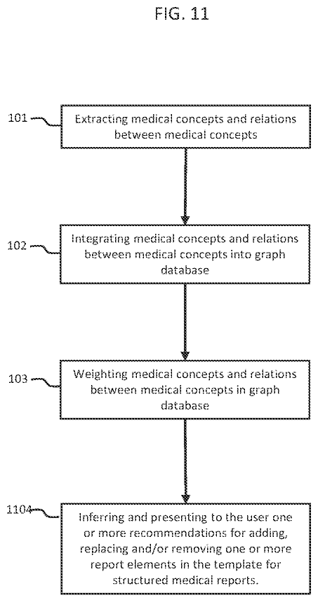

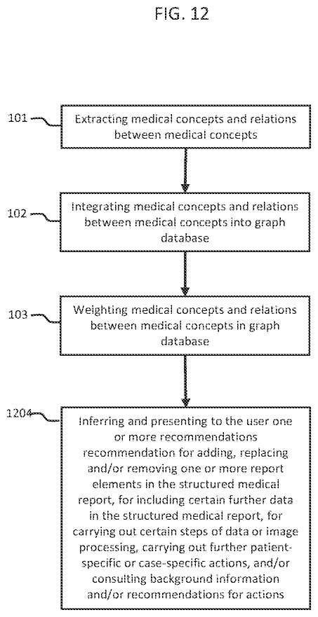

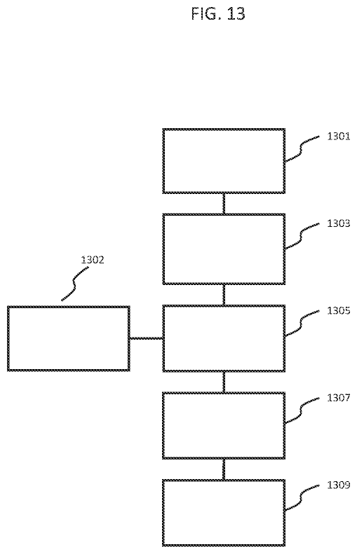

In some embodiments, illustrated in FIG. 11, when creating a report template, it may be recommended 1104 to the user to add certain elements or modules to the template, to replace them with others, or to remove them. In some embodiments, illustrated in FIG. 12, when creating a medical report, it may be recommended to the user to add certain elements or modules of the template, to replace them with others, or to remove them, e.g. as a supplement to the open template. Additionally, or alternatively, when creating a medical report, it may be recommended 1204 to the user to include certain further data in the report, e.g. associated datasets from the past examinations, to carry out certain steps of data or image (post-) processing, and/or to carry out further patient-specific or case-specific actions. The latter may also include further reporting steps. Also, it may be recommended 1204 to the user to consult certain background information/resources and/or recommendations for action, e.g. similar patient cases, guidelines, and/or scientific publications.

In order to facilitate the identification of existing concepts while composing a report template and/or a structured report, pre-existing medical language dictionaries and medical synonym dictionaries can be applied to infer most likely matches. This may be particularly useful in cases where the user is entering free-text or creating textual entries in the template or the report.

FIG. 13 illustrates an embodiment of a computer system supporting data-based clinical decision-making.

The first data storage 1301 stores the structured medical reports described above and/or templates for such reports.

The data extractor 1303 accesses the data storage and extracts medical concepts and/or the relations between the medical concepts from the structured medical reports and/or templates for such reports stored there and forwards them to the graph database manager 1305 or grants the latter access to them.

The Graph Database Manager 1305 integrates the medical concepts and the relations between medical concepts into a graph database, which is stored in a second data storage 1302. In addition, the graph database manager weights the medical concepts and the relations between medical concepts.

Based on the weighted medical concepts stored in the graph database and the relations between these concepts, the recommender 1307 infers at least one recommended action for the user. This may be one of the recommendations already discussed above.

Via the output unit, the at least one inferred recommendation is presented to the user.

The first and the second data storage 1301, 1302 can be identical. The first and the second data storage may comprise one or more solid-state drives (SSD), one or more hard-disk drives (HDD). They can be part of local data storage, a server and/or a data repository.

The data extractor 1303, the graph database manager 1305 and the recommender 1307 may be implemented by one or more processors consisting of one or more cores. They may come as independent units with their own processor(s), or they may be combined in one unit with one processor(s). Mixed forms are also conceivable, in which, for example.
the data extractor 1303 comes as one unit and the graph database manager 1305 and the recommender 1307 are combined in one unit;
the graph database manager 1305 comes as one unit and the data extractor 1303 and the recommender 1307 are combined in one unit; or
the recommender 1307 comes as a unit and the graph database manager 1303 and the data extractor 1305 are combined in one unit.

The output unit 1309 may be any device(s) suitable for visual or auditory output, in particular a monitor, glasses, ePaper, projector, or speaker.

What is claimed is:

1. A computer-implemented method for supporting data-based clinical decision-making comprising:
a. extracting medical concepts and relations between medical concepts contained in a structured medical report and/or a template for such reports, the template and/or the structured medical report comprising a data structure representing the medical concepts and relations between these medical concepts;
b. integrating the extracted medical concepts and relations between the medical concepts into a graph database;
c. weighting the medical concepts and relations between these medical concepts of the graph database according to their relevance; and
d. based on the weights of the medical concepts and relations between these medical concepts stored in the graph database, inferring and presenting to a user one or more recommendations for actions the user may take when composing report templates and/or structured medical reports.

2. The method according to claim 1, further comprising extracting from structured medical report and/or a template for such reports annotations of the medical concepts and the relations between the medical concepts, the annotations being based on a medical ontology.

3. The method according to claim 1,
wherein the data structure representing medical concepts and relations between these medical concepts comprised in the template and/or the structured medical report is chosen to have a tree structure.

4. The method according to claim 1, wherein extracting further comprises extracting attributes of the medical concepts and/or relations between the medical concepts and/or meta data.

5. The method according to claim 1, wherein weighting the elements of the graph database comprises weighting them according to the frequency of their occurrence within the templates for the structured medical reports and/or the corresponding structured medical reports.

6. The method according to claim 1, wherein weighting the elements of the graph database comprises weighting them according to one of the modality or type of examination, the template author, the country, region or language of the report or its template, and/or clinical discipline.

7. The method according to claim 1, wherein weighting the elements of the graph database comprises training the graph database based on user input data using a machine learning algorithm.

8. The method according to claim 1, comprising selecting the weights on which the recommendations are inferred based on the input parameters of the patient.

9. The method according to claim 1, wherein inferring and presenting to the user one or more recommendations comprises inferring a recommendation for adding, replacing and/or removing one or more report elements in the template for structured medical reports.

10. The method according to claim 1, wherein inferring and presenting to the user one or more recommendations comprises inferring a recommendation for adding, replacing and/or removing one or more report elements in the structured medical report, for including certain further data in the structured medical report, for carrying out certain steps of data or image processing, carrying out further patient-specific or case-specific actions, and/or consulting background information and/or recommendations for actions.

11. A computer system supporting data-based clinical decision making comprising
a first data storage storing at least one structured medical report and/or a template for such reports, the template and/or the structured medical report comprising a data structure representing medical concepts and relations between the medical concepts;
a data extractor that extracts medical concepts and relations between medical concepts contained in the stored structured medical report and/or the template for such reports;
a graph database manager that
a. integrates the extracted medical concepts and relations between the medical concepts into a graph database stored to the first or a second data storage; and
b. weights the medical concepts and relations between the medical concepts integrated into the graph database;
a recommender that infers one or more recommendations for actions a user may take, based on the weights of the medical concepts and relations between these medical concepts stored in the graph database; and
an output unit that presents the inferred recommendations to the user.

12. A computer readable storage medium having stored there a program product supporting data-based clinical decision-making, wherein, when executed by a computer processor, the computer processor is caused to carry out steps comprising:
a. extract medical concepts and relations between medical concepts contained in a structured medical report and/or a template for such reports, the template and/or the structured medical report comprising a data structure representing the medical concepts and relations between these medical concepts;
b. integrate the extracted medical concepts and relations between the medical concepts into a graph database;
c. weight the medical concepts and relations between these medical concepts of the graph database according to their relevance; and
d. based on the weights of the medical concepts and relations between these medical concepts stored in the graph database, infer and presenting to a user one or more recommendations for actions the user may take when composing report templates and/or structured medical reports.

13. The storage medium according to claim 12, wherein the computer processor is further caused to extract from structured medical report and/or a template for such reports annotations of the medical concepts and the relations between the medical concepts, the annotations being based on a medical ontology.

14. The storage medium according to claim 12, wherein the data structure representing medical concepts and relations between these medical concepts comprised in the template and/or the structured medical report is chosen to have a tree structure.

15. The storage medium according to claim 12, wherein extracting further comprises extracting attributes of the medical concepts and/or relations between the medical concepts and/or meta data.

16. The storage medium according to claim 12, wherein weighting the elements of the graph database comprises weighting them according to the frequency of their occurrence within the templates for the structured medical reports and/or the corresponding structured medical reports.

17. The storage medium according to claim 12, wherein weighting the elements of the graph database comprises weighting them according to one of the modality or type of examination, the template author, the country, region or language of the report or its template, and/or clinical discipline.

18. The storage medium according to claim 12, wherein weighting the elements of the graph database comprises training the graph database based on user input data using a machine learning algorithm.

19. The storage medium according to claim 12, wherein the computer processor is further caused to select the weights on which the recommendations are inferred based on the input parameters of the patient.

20. The storage medium according to claim 12, wherein inferring and presenting to the user one or more recommendations comprises inferring a recommendation for adding, replacing and/or removing one or more report elements in the template for structured medical reports.
